(19) United States
(12) Patent Application Publication
(10) Pub. No.: US 2008/0215028 A1
(43) Pub. Date: Sep. 4, 2008

(54) HIGHLY-ADJUSTABLE, FITTED CLOTH DIAPER

(76) Inventors: KEREN BROWN, NAMPA, ID (US); TRACY MILJKOVIC, NAMPA, ID (US)

Correspondence Address: PEDERSEN & COMPANY, PLLC, P.O. BOX 2666, BOISE, ID, 83701, US

(21) Appl. No.: 11/945,264

(22) Filed: Nov. 26, 2007

Related U.S. Application Data
(60) Provisional application No. 60/860,933, filed on Nov. 24, 2006.

(51) Int. Cl. A61F 13/15

(52) U.S. Cl. 604/385.15; 604/385.01; 604/386

(57) ABSTRACT

A cloth diaper is fitted, washable and reusable, and highly effective in reducing or preventing leaks. The diaper comprises fabric preferably on both the outermost surface and the inner most surface that is stretchable, soft, and has hydrophilic and wicking properties. The diaper comprises an inner absorbent layer(s) between said hydrophilic and wicking fabric layers. A preferred continuously-adjustable fastening system may be used to adapt the diaper to fit a wide range of sizes of infants and toddlers, for example, from birth up to four years old. The fastening system allows a large amount of the front side of the diaper to be folded inside the diaper, significantly changing the overall size of the diaper to accommodate small infants. Even with the large folded-in flap, soft, wicking fabric is maintained against the child's skin, and the fastener system portion that is also folded inward does not interfere with absorbency. The preferred “V-shaped” fastener extends far down on the front side of the diaper, most of the way to the “crotch” area of the diaper, and also has wings/extensions from the top ends of the arms of the “V”, thus, allowing fastening of the diaper all along the length of each of the arms of the “V” and also out along the transverse length of the wing/extensions.

This application claims priority of Provisional Application 60/860,933, filed Nov. 24, 2006, the entire disclosure of which is incorporated herein by this reference.

### FIELD OF THE INVENTION

The invention relates to non-disposable diapers, and particularly to cloth diapers that are fitted and include fasteners that are used to adjust the diaper to various sizes of infants.

### RELATED ART

Given the stressful and busy lives of parents and infant caretakers, it is desirable to have form-fitting and adjustable diapers for infants and for older toddlers. Given the environmental impact of disposable diapers, it is desirable to provide a well-fitting, adjustable, washable and reusable diaper. Many attempts at these goals are represented in the prior art, for example, in the following patents: Redfern (U.S. Pat. No. 4,402,690, issued Sep. 6, 1983); Sorenson, et al. (U.S. Pat. No. 4,801,298, issued Jan. 31, 1989); Coates (U.S. Pat. No. 5,137,526, issued Aug. 11, 1992); Zehner, et al. (U.S. Pat. No. 5,366,453, issued Nov. 22, 1994); Coates (U.S. Pat. No. 5,725,518, issued Mar. 10, 1998); LaVon, et al. (U.S. Pat. No. 6,932,800, issued Aug. 23, 2005); Karlsson (Application Publication 2002/0042602, published Apr. 11, 2002); Abrahamsson (Application Publication 20021/0091366, published Jul. 11, 2002); Nakaoka, et al. (Application Publication 2002/0138061, published Sep. 26, 2002); Erdman (Application Publication 2007/0073258, published Mar. 29, 2007); Coates (Application Publication 2005/0022291, published Feb. 2, 2005); and Baskeiville (Application Publication 2004; 0153045, published Aug. 5, 2004).

Still, there is a need for a reusable, well-fitting, cloth diaper that is not prone to leaking, and that is highly adjustable and highly durable. The present invention meets these needs.

### SUMMARY OF THE INVENTION

Objects of the invention include providing a diaper that is fitted, washable and reusable, highly unlikely to leak, and extremely durable. The invented diaper comprises fabric preferably on both the outermost surface and the inner most surface that is stretchable, soft, and has hydrophilic and wicking properties. The invented diaper comprises an inner absorbent layer(s) between said wicking fabric layers. This results in the preferred hydrophilic, wicking fabric becoming dryer as time passes after wetting, as liquid is wicked into the highly-absorbent layers between the outer layers of the diaper. As a result, the outermost fabric layer of the diaper becomes very dry to the touch within a few minutes.

A fastening system is provided that may be used to adapt the diaper to fit a wide range of sizes of infants and toddlers, for example, from birth up to four years old. The preferred fastening system adjusts the width (waist size) and height of the diaper continuously within wide ranges, making an effective and comfortable fit possible without the use of incremental fasteners such as snaps. The fastening system allows a substantial amount of the front side of the diaper to be folded inside the diaper, significantly changing the overall size of the diaper to accommodate small infants. In this much-reduced-size configuration, a substantial amount of the outermost layer of the diaper is folded inward against the child's skin, but, due to the structure of the diaper, soft, wicking fabric is maintained against the child's skin, and the fastener system does not interfere with absorbency. The preferred “V” fastener component of the fastener extends far down on the front side of the diaper, to allow fastening of the diaper even when about seven inches of the front side of folded inward. The preferred “V” fastener component also has “wings” of fastener extending out from each upper end of the V-fastener, which allow substantial adjustment of the waist size of the diaper for the largest of children using the diaper. The preferred “V” fastener arms and wings are integral portions of the preferred one-piece “V,” so there are no seams or ridges along the “V” that could irritate or scrape the infant.

The preferred V-fastener is a “hook compatible fabric” rather than a conventional hook and loop strip with rigid or semi-rigid backing such as Velcro™. The hook-compatible fabric is soft, and has soft edges, so that it will rest against the infant's skin, in the reduced-size configuration, without irritating, scraping, or poking the skin. In other words, the preferred loop material for the V-fastener is made of soft, low-pile loop fabric that will not irritate the baby's skin when folded in to fit a newborn. Further, when the front of the diaper is folded in and the hook tabs are fastened to the loop V-fastener, the baby is still able to wet on the hydrophilic wicking fabric that is between the widely-spaced arms of the “V”. Thus, the loop “V” closure system may provide a snug, comfortable fit from birth to 4 years old.

The preferred pattern of the diaper includes a contour cut or “scoop” in the top edge of the back of the diaper, which allows the diaper to fit comfortably at baby's lower back and reduces the need for excessive elastic to insure a snug, leak free fit. This preferred scoop in the pattern also eliminates the typical pooch of fabric at the back of cloth diapers, making it more trim under clothing.

Elastic is provided in the back of the diaper but is gentle and minimal. Preferably, a fairly loose elastic is slightly gathers only the scoop area of the back edge of the diaper. This short length of elasticized edge is enough to gently pull the right and left portions of the back edge toward the scooped area, so that overall the back edge fits to the back of the infant to blocking leaks from the back, but without squeezing the waist too tightly and without gouging into the infant's skin. Thus, the back elastic does not irritate baby's sensitive skin by over-gathering the fabric into deep, tight gathers that tend to mark the skin at pressure-points.

Elastic is provide substantially around the leg holes formed by diaper, that is, around most of the leg hole formed by the diaper in the reduced-sized configuration, and around about at about the bottom half of the leg hole formed by the diaper in the full-size configuration. The elastic is thus positioned to be gentle enough for wide legs on toddlers and still close the diaper snugly around newborn legs to prevent leaks. The edges of the diaper that form the leg hole lack of top stitching over the leg opening elastic, which reduces gathering of the fabric into deep and/or tight gathers that would tend to mark the skin at pressure points. Hence, the leg holes and their elastic are more gentle on baby skin than conventional elastic-gathered garments.

### BRIEF DESCRIPTION OF THE DRAWINGS

FIG. 1 is a perspective, exploded view of one embodiment of the invented diaper, with cut-out layers of fabric aligned with each other but prior to sewing of the diaper. The preferred elastic and preferred fasteners are not shown in this view.

FIG. 2 is perspective view of a preferred embodiment of the invented diaper, fastened for a medium-sized toddler, for example.

FIG. 3 is a front view of the embodiment of FIG. 2, with the fastener system unfastened but with the front of the diaper in full-size configuration, that is, without the front side of the diaper being folded over.

FIG. 4 is a front view of the embodiment of FIGS. 2 and 3, with the fastener system unfastened and the two corners of the diaper spread out and flat, prior to being wrapped around the child.

FIGS. 5-7 are a front perspective, a top view, and a rear perspective, respectively, of the embodiment of FIGS. 2-4, wherein about half of the front side of the diaper has been folded inward and the fastener system has been fastened for a smaller, but probably not a newborn, infant. The preferred elastic systems for the top, rear edge of the diaper and the leg holes are shown in the rear view of FIG. 7.

FIG. 8 portrays an especially-preferred embodiment of the invented diaper, unfastened and flat, wherein the inner panel(s) of the diaper is/are in view.

FIG. 9 portrays the embodiment of FIG. 8, unfastened and flat, with the outer panel(s) in view.

FIG. 10 is a front view of the embodiment of FIGS. 8 and 9, folded and fastened for a newborn, wherein the front side of the diaper has been folded inward to greatly reduce the height, which may be thought of as the vertical distance from the bottom of the “crotch” area to the upper edge of the diaper (from bottom to top of diaper in direction from bottom to top of the sheet of paper).

FIG. 11 is a side view of the diaper in its reduced-size configuration of FIG. 10.

FIG. 12 is a schematic cross-sectional view of the diaper in its reduced-size configuration of FIGS. 10 and 11, showing how the front side of the diaper has been folded inward.

FIG. 13 is a front view of the diaper of FIGS. 8-12, fastened in a nearly-full-size configuration, for example, for a medium-size toddler.

FIG. 14 is a side view of the diaper of FIG. 13.

FIG. 15 is a schematic cross-sectional view of the diaper in its nearly-full-size configuration of FIGS. 13 and 14, showing how the front side of the diaper is Lm-folded so that the diaper is at its maximum height, and showing how the fasteners are fastened at nearly their maximum waist-width/circumference. The view is taken along the line 15-15 in FIG. 13.

FIG. 16 is a front view of the diaper of FIGS. 8-15, wherein the diaper is fastened at its maximum height and width, with the fasteners fastened on the “wings” to maximize the waist width/circumference.

### DETAILED DESCRIPTION OF THE PREFERRED EMBODIMENTS

Referring to the Figures, there are shown two, but not the only, embodiments, of the invented fitted, highly-adjustable cloth diaper.

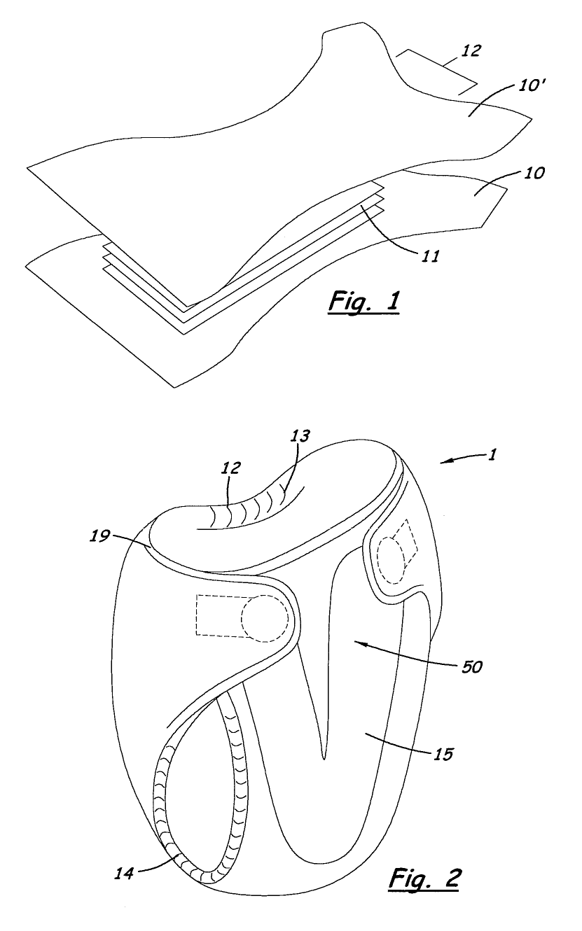

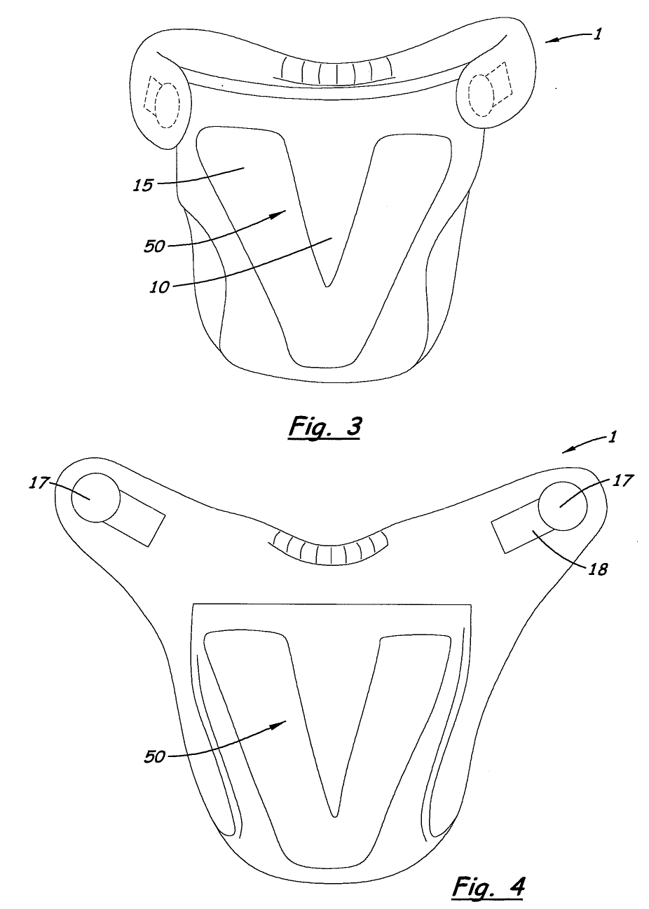

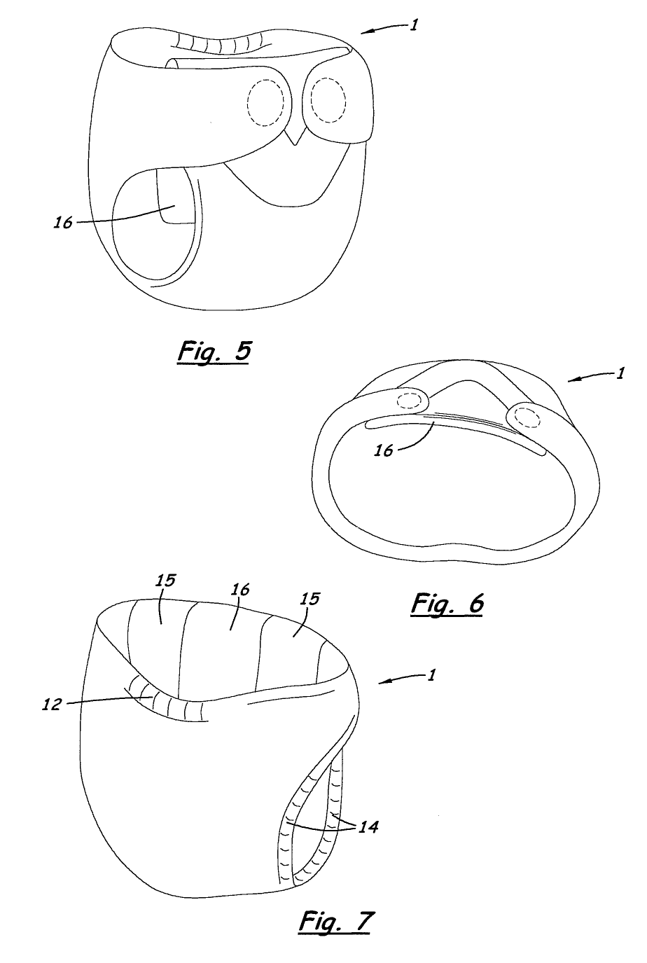

The preferred diapers 1, 100 comprise very stretchy, soft, fabric panels 10. 10′ with hydrophilic and wicking properties, wherein said panels 10 are the outermost and innermost panels of the diaper, as shown to best advantage in FIG. 1. While wicking fabrics serve the wicking function to different degrees, the especially-preferred fabric for panels 10, 10′ has the capacity to hold water before it is transported to the diaper's inner absorbent layer(s) 11, but quickly wicks to its inner surface and to said inner absorbent layer(s) 11. This results in the outer surfaces of the hydrophilic fabric panels 10, 10′ actually becoming dryer as time passes after the infant wets. As a result of the combination of the hydrophilic and wicking panels and the inner-super absorbent layer(s) 11, the outer surfaces of the preferred panels 10, 10′ are left so dry that it is hard to tell, after about 10 minutes, that the fabric was previously wet. The especially-preferred wicking fabric for panels 10, 10′ may be selected from various moisture-transport fabrics, such as are known in the sporting and recreational goods fields, and are typically hypoallergenic, synthetic materials, for example, hypoallergenic 100% polyester.

The super absorbent inner layer(s) 11 is/are provided in between panels 10, 10′. Preferred layers 11 may be composed of any number of (preferably several) rectangular fabric pieces that are attached to the inside panel 10 of the diaper and sewn in permanently and out of sight when the diaper is finished. Thus, panels 10, 10′ completely cover and surround layers 11. Layers 11 preferably are made of fabrics that will reduce bacterial growth. Preferably, layers 11 are made of synthetic/non-natural fibers that are preferably hypoallergenic and that minimize bacterial growth inside the diaper.

The preferred diaper structure of entirely synthetic fabrics tends to be safe for infants' skin, clean and unlikely to harbor bacteria, and durable enough to be washed repeatedly and vigorously without quick damage or degradation. The preferred embodiments do not degrade when washed an average of 2-3 times per week for a minimum of 3 years, which is very beneficial for families or institutions that want a given diaper to last during use on an infant over many months and even up to 4 years of age.

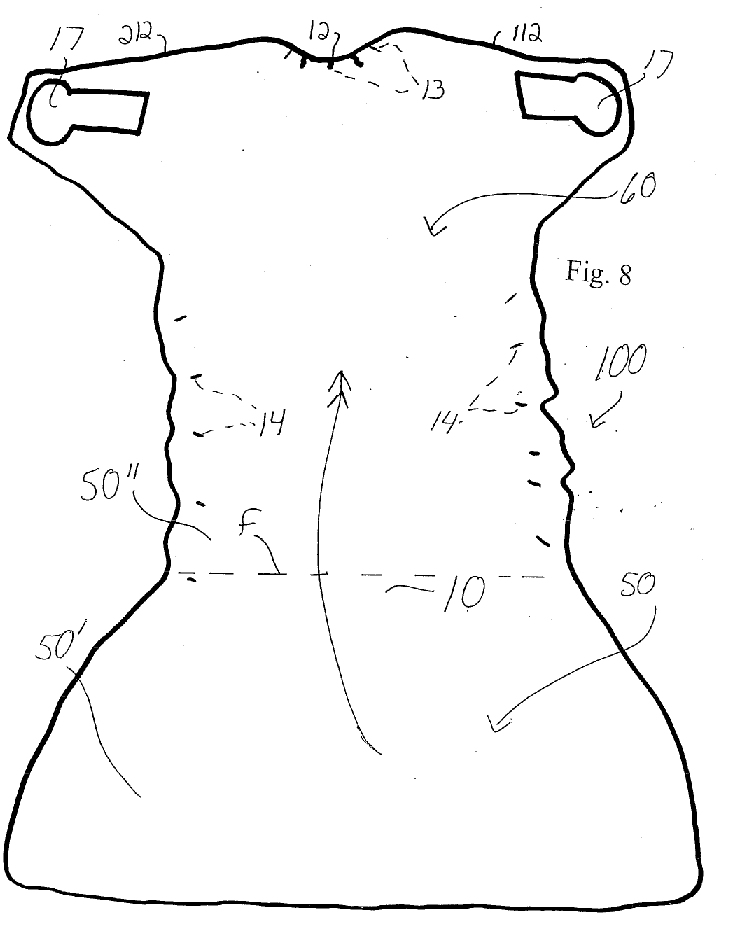

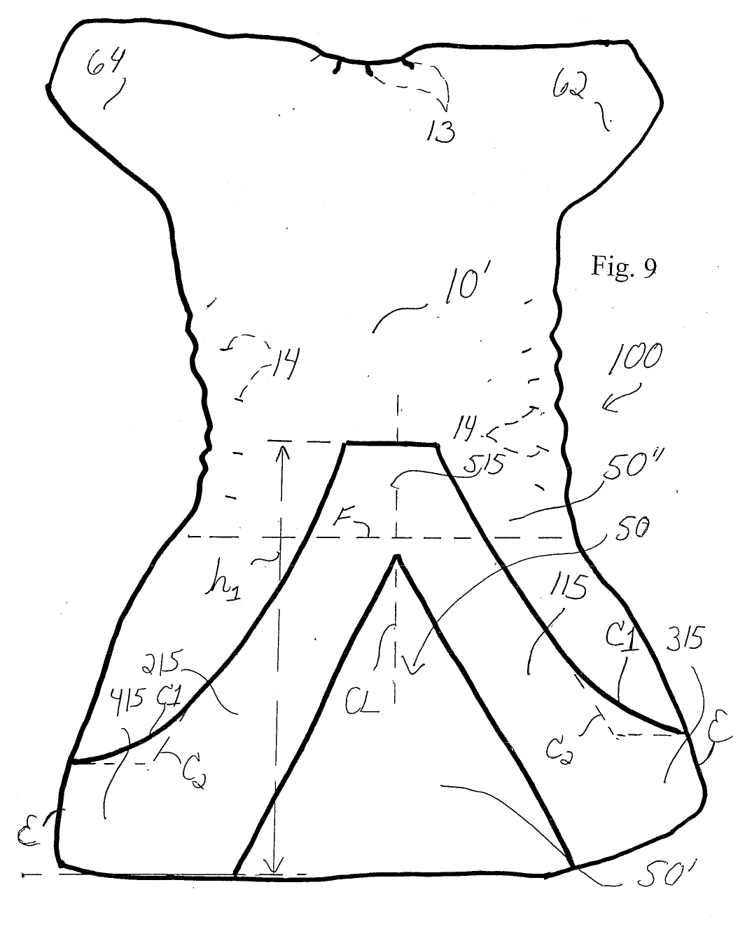

A contour or “scoop” 12 cut in back of pattern (in the upper, back edge of the diaper as it will be worn) allows diaper 1, 100 to fit comfortably at baby's lower back, reducing the need for elastic to insure a snug, leak free fit. This scoop 12 in the pattern also eliminates the typical pooch of fabric at the back of cloth diapers, making it more trim under clothing. Back elastic 13 is included, preferably, only along the length of the upper, back edge of the diaper that is scooped inward toward the center of the diaper (scoop 12). The elastic is gentle and minimal, that is, pulling only gently (and not tightly and strongly) on the adjacent edge portions 112, 212, while still being effective in blocking leaks because the fabric at scoop 12 gathered to be against the infant's skin. The elastic 13 in the area of scoop 12 does not irritate baby's sensitive skin by over-gathering the fabric. The call-out lines for elastic 13 is shown in dashed lines pointing to the gentle gathers in FIGS. 8 and 9, to represent the fact that the elastic 13 is preferably hidden inside the diaper, between panels 10, 10′. Preferably, only chlorine and chemical resistant swimsuit grade elastics are used for elastic 13 (and for elastic 14, discussed below) in order to insure maximum durability when exposed to the diaper pail solutions and detergents from washing diapers an average of 2-3 times per week.

Leg elastic 14 is positioned along the side edges of the diaper 1, 100, and is also adapted to be gentle, rather than strongly-pulling on the edge areas of the leg holes. It is desirable for elastic 14 to be gentle enough for wide legs on toddlers and still close the diaper snugly around newborn legs to prevent leaks. Elastic 14 is shown in FIGS. 8 and 9, by dashed lines pointing at the gentle gathers in the leg hole area, to indicate that the elastic 14 is preferably hidden inside the diaper, between panels 10, 10′. There is preferably no top stitching over the leg opening elastic, which reduces gathering in the fabric (making it be bunched or billowed together in the leg area but not pulled together in tight, deep gathers). Thus, the elastic 14 and the leg openings are more gentle on baby skin that deeply-gathered and/or top-stitched edges, and will tend not to create the red markings and impressions in infants' skin that are common with other elastic garments, diaper covers, or fitted diapers.

The fastening system preferably includes a “V” shaped piece of loop fabric (or “hook compatible fabric” 15), that adjusts the width and height of the diaper, making an effective and comfortable fit possible without the use of limited snap or other incremental settings/fasteners. The loop “V” is made of soft, low-pile loop fabric that will not irritate the baby's skin when folded in to fit a newborn. It is preferred that the V-shaped piece of fabric be nylon knitted loop fabric, or other fabric that has a soft “hand” and no laminated or other stiffener or stiff backing. While a conventional loop fastener such as Velcro™ might work for connecting to the hook portions of the preferred fastening system, said conventional loop fastener strips have stiff backings with edges that can be harsh, abrasive, and/or irritating to skin. Therefore, a hook-compatible material (a term known in the fabric field) is desired rather than a conventional hook-and-loop strip comprising loops and rigid or semi-rigid backing and edges.

Preferably, the V-shaped fastener 15 has two aims 115, 215 that extend upwards from the point of the “V” at an angle to each other of about 55-75 degrees (and more preferably about 65 degrees). The top edge of each arm is about 6-7 inches vertically (h1) from the bottom of the “V” (see FIG. 9). At the upper end of each of the arms is a transverse wing 315, 415, that extends outward away from the centerline of the diaper, to extend the width of the “V” at the top of the “V”. These wings 315, 415, as will be discussed in more detail below, allow the waist width (circumference) to be widened for large toddlers, for example, up to age four years.

By using the preferred hook-compatible fabric, without stiffener or stiff edges, as the fastener, and by forming the V fastener to have wide-spread arms and to extend down far along the front side 50 of the diaper, said front side 50 may be folded over, inward toward the infant and toward the rear side 60 of the diaper, and fastened to configure the diaper into a newborn or other very small child diaper. The flattened diaper (see FIGS. 8 and 9) is approximately 17.5 inches vertically (top to bottom of sheet in FIGS. 8 and 9), approximately 7 inches wide transversely across the diaper from leg hole edge to leg hole edge, and approximately 14 inches across the diaper from tab 17 to tab 17. The front side 50 may be folded over toward the infant at any place along the V that leaves at least an inch or more of the V exposed for attachment of the hook tabs 17 to the V fabric 15. As shown in FIGS. 8 and 9 to best advantage, the maximum amount of front side 50 that may be folded is represented by maximum fold line F. As portrayed by the double-headed arrow in FIG. 8, the front side 50 of the diaper would be folded toward side 60 in FIG. 8, with the front side therefore becoming folded/creased at fold line F.

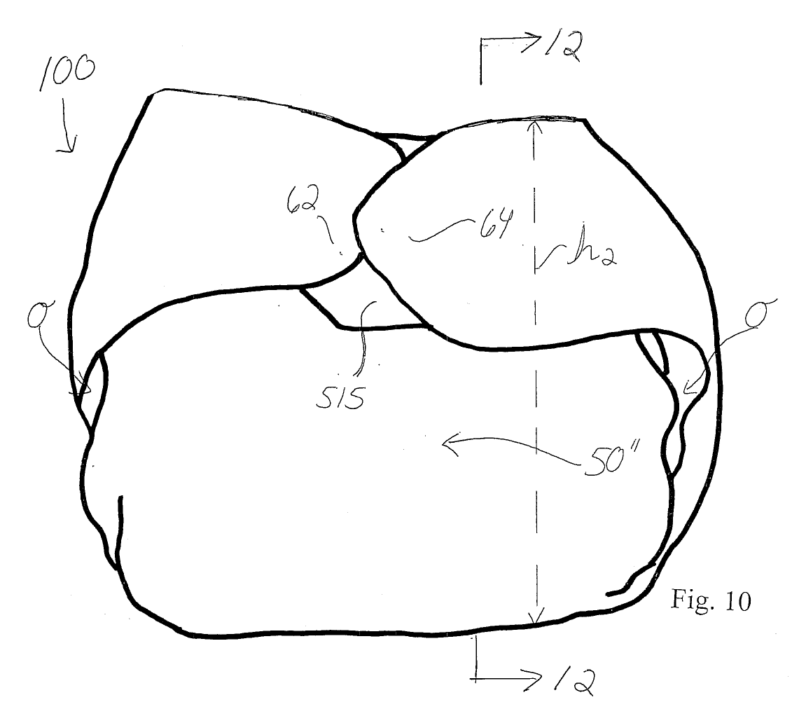

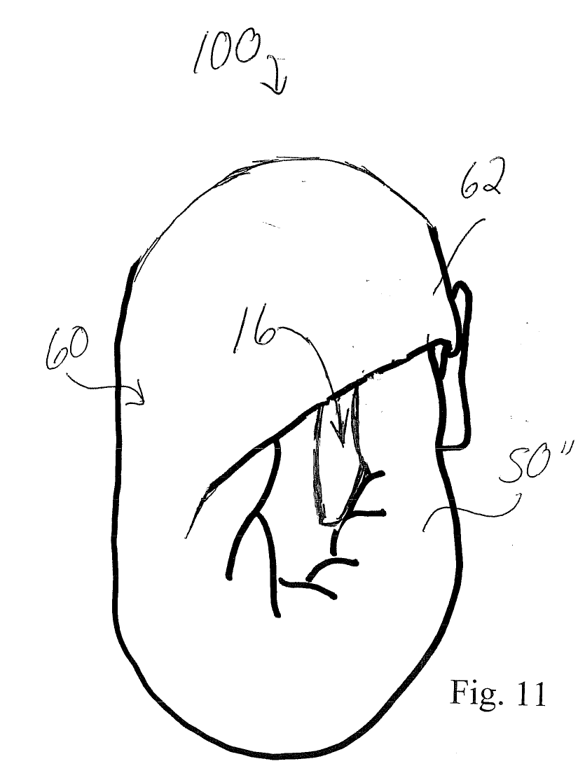

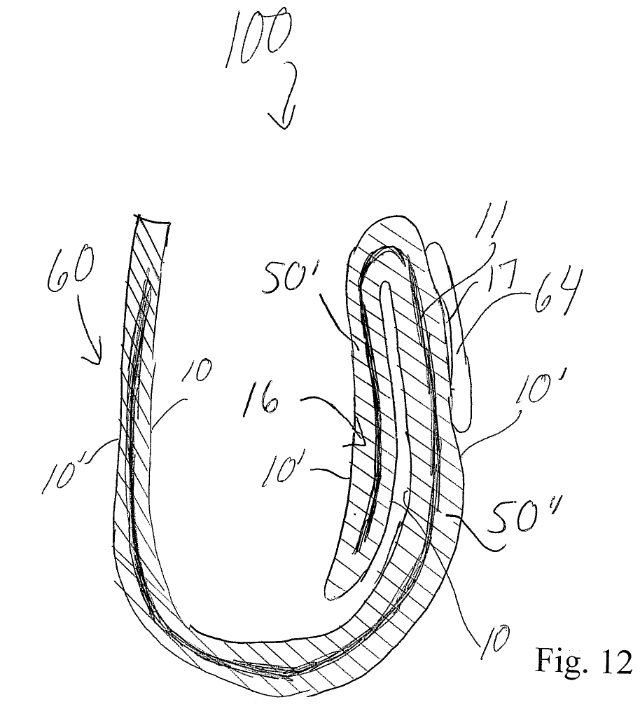

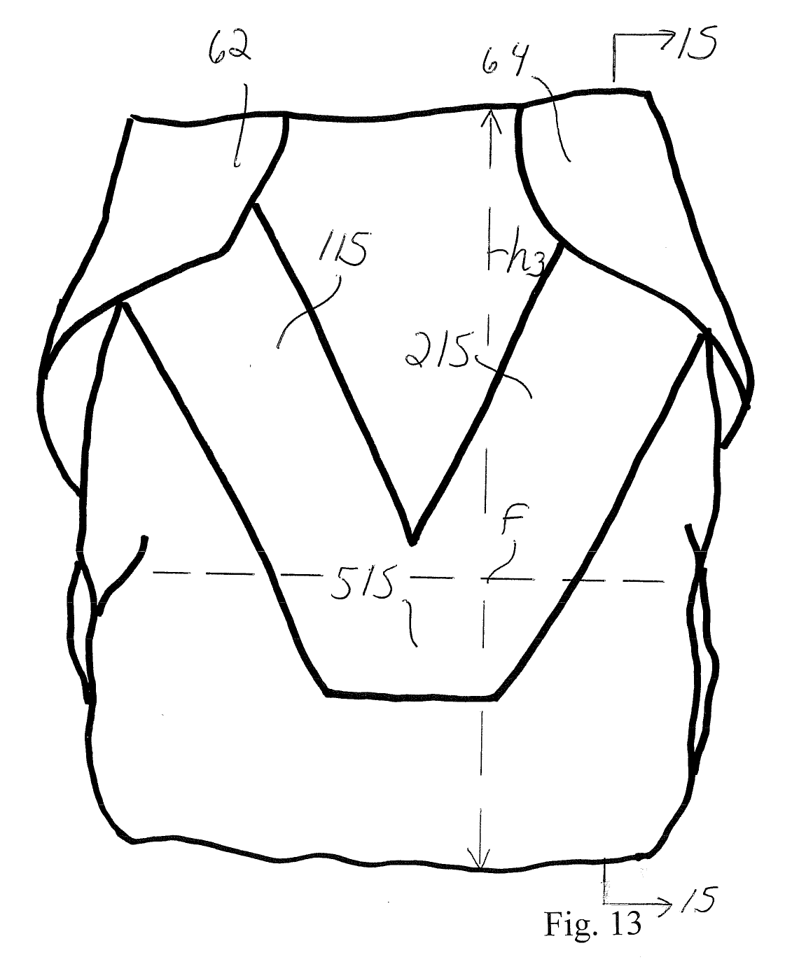

FIGS. 10 and 11 illustrate the diaper 100 in its maximum-folded, minimum height configuration, wherein the front side 50 has been folded inward to create flap 16, wherein flap will rest against the infant's skin. The flap 16 adds another thick layer/layers (of wicking material with absorbent material within), to increase overall absorbance in the front area of the diaper. The diaper 100 is designed so that the flap 16, in the maximum-folded, minimum height configuration, will extend at least 5 inches into the interior space of the diaper (where the infant is), and preferably at least 6 inches, with the fastener system still working well to securely hold the diaper on the infant. This long extension of the flap not only allows a large adjustment of the size of the diaper, but the extra absorbency of the extra flap in the interior space may be particularly beneficial. The extra absorbency in the flap area is very beneficial for boy babies, but will be beneficial for all babies in view of the excellent fit that this substantial fold-over feature affords.

In FIG. 10, one may see the “ears” 62, 64 wrapping around to the front of the diaper, so that the hook tabs 17 can contact and hook onto the lower region 515 of the V fastener near the point of the V. Leg openings 0 are relatively small and gently-gathered for leak prevention. One may see in FIG. 10 that the bottom end/point of the V fastener is not fairly high up on the front of the diaper, because the over height h2 of the diaper has been reduced by said folding a large portion 50′ (most of the front side 50) behind the lower region 50″ of front side 50. See FIG. 1 for a side view that shows a portion of the folded over portion 50′ through the right leg hole. See FIG. 13 for a schematic cross-section of the diaper configuration in FIG. 10, viewed along line 13-13.

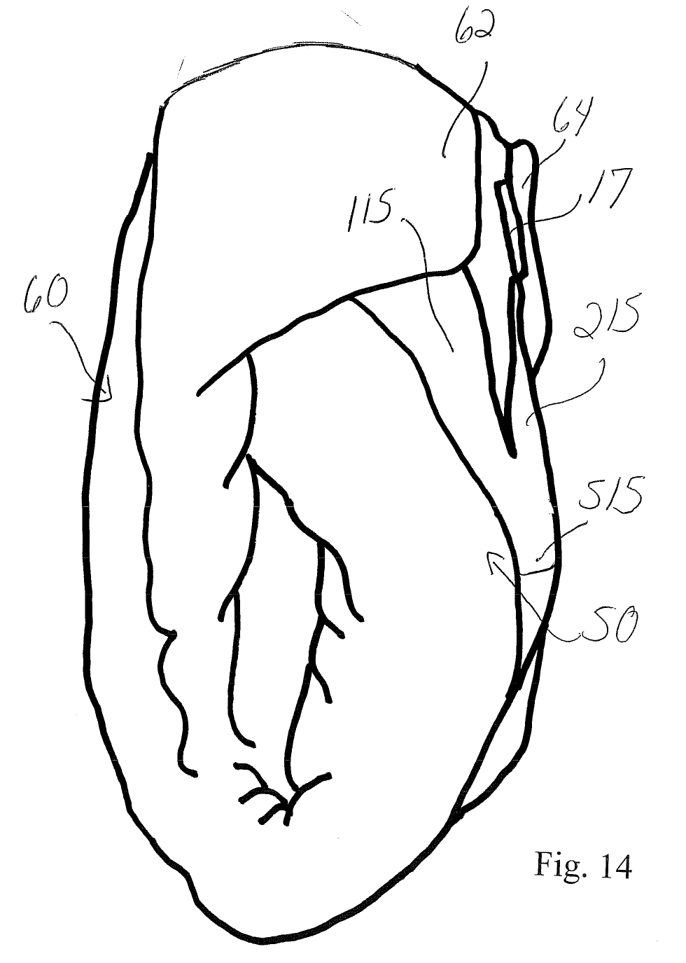

When the front side of the diaper is folded and the hook tabs 17 are fastened, the baby is still able to wet on an area of hydrophilic wicking fabric of the flap 16, between the arms of the V of the flap 16. This folding-over of the front side portion 50′ is more than a mere small tuck-in or small fold-over to prevent wetting of the infant's belly-button area; it is a major change in the size, and particularly the height of the diaper. For example, the diaper configured in its full height (see FIGS. 13-16, discussed below) may be about 8-9 inches high (h3), but the diaper in its fully-reduced height may only be about 4-6 inches, and typically about 5 inches high. Therefore, the height of the diaper may be reduced by about 30-50 percent, and preferably at least 30 percent.

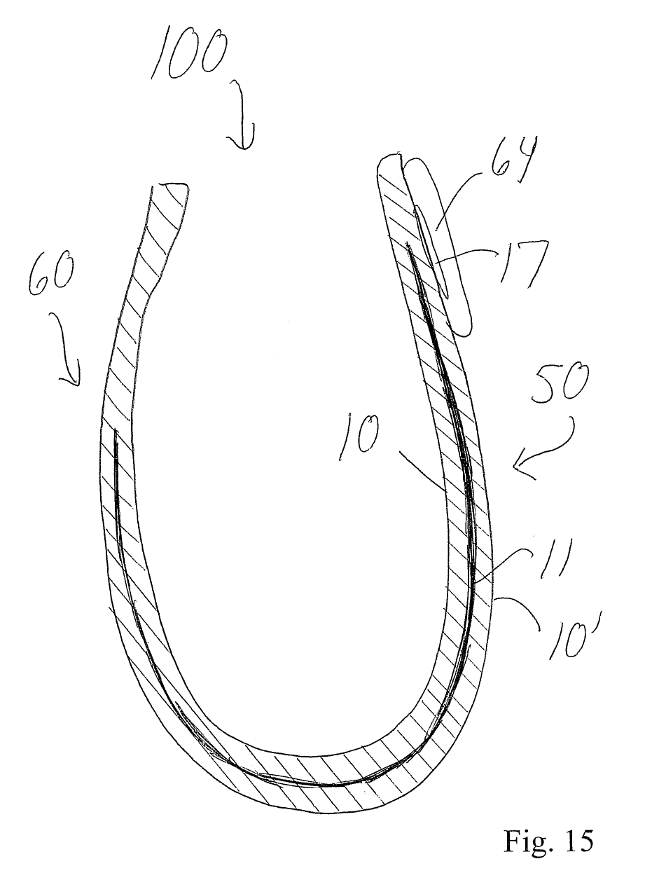

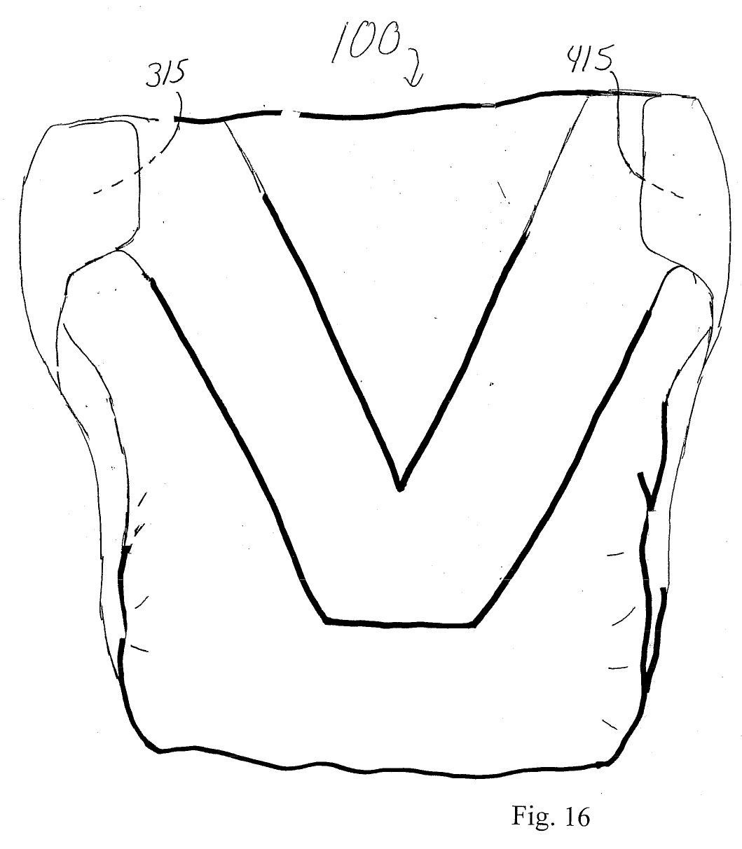

FIGS. 13-15 illustrate the diaper 100 in a full-height (but not full waist circumference) configuration. The front side 50 is not folded over (not folded over either inward or outward) and the ears 62, 64 are wrapped around to the front of the diaper so that hook tabs 17 attach to the top region of each arm 115, 215 (but not the wings 315, 415). FIG. 15 is a schematic cross-section, viewed along the line 15-15 in FIG. 13.

FIG. 16 illustrates the diaper 100 in full height and full waist circumference configuration, such as may be used for a toddler of age four years, for example. The ears 62, 64, and their hook tabs 17 (not shown in this figure, but understood from FIG. 8, for example) are wrapped around the diaper just enough to contact and attach to the wings 315, 415 that extend transversely (horizontally in FIG. 16) from the top ends of the arms 115, 215 of the V fastener. Because the outer edges E of the preferred wings 315, 415 are about 4.5-5 inches from the centerline CL of the preferred V fastener (see FIG. 9), one may see that the maximum increase in waist circumference may be about 7-9 inches (taking into account that the tabs 17 will not typically both be on the centerline and will not typically be on the edges E). The wings 315, 415 may have a gradually-curved transition from the main arms 115, 215 of the V fastener, or may angle to the right and left (transversely outward, as discussed above) without the gentle curve represented at C1 but instead at a sharp angle represented by dashed line C2 in FIG. 9.

Thus, the “V” fastener 15 allows the diaper to adjust the diaper's width and height freely and continuously, which is not possible with a fixed, incremental snap-based adjustment system. The loop “V” closure system provides a snug, comfortable fit from birth to potty training (approx. 5-35 lb.).

The use of hook coins (die-cut circles of hook tape) is preferred for the hook tabs 17, instead of square pieces of hook assures that sharp corners do not peel up and scratch the baby. Also, laundry tab covers (rectangles of loop tape) 18 are preferably sewn next to the tabs 17 in order to provide a contact point for the tabs 17 when washing and drying the diaper. By attaching the tabs 17 to their laundry tab covers, the hooks of the tabs 17 are kept clean of lint and fabrics are free from snagging on open hooks.

The entire diaper, excluding the leg openings. is preferably top stitched in order to create a trim, finished diaper that is more durable due to the second set of stitches. Threads designed for outdoor wilderness sports apparel (such as those used in skiing and boating) are used in order to insure the most durable construction possible.

The preferred, unique V-shaped hook-and-loop fastening system allows the diaper to adjust in width and depth at the same time, and in a continuous, rather than incremental way. This system also eliminates the restrictions and potential discomfort of a limited, snap-based, adjustable fastening system. The range of adjustability provided by the diaper eliminates the need for purchasing multiple sizes of fitted, cloth diapers as a baby grows, providing economic and ecological benefits. The diaper will adjust to fit the majority of babies from birth through potty training (approx. 5-35 lb.).

The durable materials used in the preferred diaper eliminate the need for special care or line-drying. The shape of the diaper is form-fitting, and becomes more form-fitting as a baby grows and increases in mobility. The hook and “hook compatible fabric” fastening system is extremely easy to use. The basic functions of a diaper (wicking liquid away from the baby's skin and absorbing it) are heightened by the materials used in the diaper. No separate diaper “cover” is needed, and parents and caregivers should find the preferred diapers easy to use, wash, dry, and reuse.

### OPERATION

To place the diaper on a larger baby or toddler, the parent or caregiver fastens the hooked tabs 17 to the upper portion of the V-shaped section 15 of looped fabric on the front of the diaper. To place the diaper on smaller babies, the parent or caregiver folds the front panel of the diaper down inside the diaper, and attaches the hooked tabs 17 to the lower portion (as low at the point of the V) of the looped fabric. The V-shape of the looped fabric allows the primary fabric (panel 10′) of the diaper to absorb liquid when it is folded down inside the diaper, as preferably both panels 10, 10′ are the same fabric. The looped fabric of the V fastener, and also panels 10, 10′ are soft and do not irritate a baby's skin.

Although this invention has been described above with reference to particular means, materials and embodiments, it is to be understood that the invention is not limited to these disclosed particulars, but extends instead to all equivalents within the broad scope of the following claims.

1. A fitted cloth diaper comprising:
a front side and a rear side and an interior space between the front side and the rear side for receiving an infant in the diaper;
wherein the front side comprises a V-shaped fastener of loop-containing fabric that extends from at or near a top edge of the front side to at or near a bottom end of the front side, said V-shaped fastener having two generally vertical arms that diverge upward at an angle to each other of 55-75 degrees, wherein each of said arms has an extension that extends away from a centerline of the V-shaped fastener to extend horizontally outward to outermost edges of an upper region of the front side;
wherein the rear side comprises a plurality of hooked fabric tabs that are connectable to the V-shaped fastener;
wherein said diaper is adjustable in size by folding said upper region of the front side in toward the rear side to create a flap that extends downward inside the diaper interior space a distance of at least 5 inches, and wherein, when said flap extends down into the interior space, said hooked fabric tabs extend to connect to a lower region of the V-shaped fastener to secure the diaper on the infant.

2. A diaper as in claim 1, wherein the rear side has an upper edge that has a scoop-shaped recess that comprises elastic.

3. A diaper as in claim 1, wherein said V-fastener loop-containing fabric has no stiffened backing.

4. A diaper as in claim 3, wherein said V-fastener loop-containing fabric has no stiff longitudinal edges.

5. A diaper as in claim 1, wherein said hooked tabs are circular.

6. A diaper as in claim 1, further comprising tab covers extending from the hooked tabs and adapted to bend over and attach to said hooked tabs for protecting the hooked tabs during washing and drying.

7. A diaper as in claim 1, wherein the entire upper region of the front side folds inward into the interior space along a horizontal fold line that extends through the V-shaped fastener near a lower-most point of the V-shaped fastener.

8. A diaper as in claim 1, wherein the entire diaper is synthetic fabric.

9. A diaper as in claim 1, wherein the diaper has two leg openings into the interior space of the diaper, and elastic around a portion of each leg opening, and wherein the diaper comprises no top-stitching around any portion of the leg openings.

10. A diaper as in claim 1, wherein the V-shaped fastener arms are each 6-7 inches long, and the hooked tabs adhere to the V-shaped fastener at any point continuously along the lengths of the arms.

11. A diaper as in claim 1, wherein said extensions extend horizontally outward 4.5-5 inches from said centerline on opposite sides of said centerline, and wherein said hooked tabs adhere to said extensions at any point along the extensions from a location wherein the extension extend from the arms to near said outermost side edges of the upper region of the diaper.

12. A diaper as in claim 11, wherein said arms and said extensions of the V-shaped fastener are integral portions of a single-piece of loop-containing fabric.

13. A diaper as in claim 1, wherein the diaper comprises an outermost panel of fabric and in innermost panel of fabric that are made from the same material, so that, when the upper region is folded over into the interior space, the surface of the flap contacting the infant is the same fabric as the interior fabric of the rear side of the diaper.

14. A diaper as in claim 7, wherein the diaper is adjustable in size by said folding along said horizontal fold line to fit a newborn infant up to a four year old toddler.
